(19) United States
(12) Patent Application Publication
(10) Pub. No.: US 2009/0012177 A1
(43) Pub. Date: Jan. 8, 2009

(54) Treatment of psychiatric disorders using entacapone, tolcapone and other COMT inhibitor or MB-COMT inhibitor drugs

(76) Inventors: Rahim Shafa, Lexington, MA (US); Hamid Mostafavi Abdolmaleky, Brookline, MA (US)

Correspondence Address: Rahim Shafa, 8 Fairland Street, Lexington, MA, 02421, US

(21) Appl. No.: 11/689,682

(22) Filed: Mar. 22, 2007

Related U.S. Application Data
(60) Provisional application No. 60/785,097, filed on Mar. 23, 2006.

(51) Int. Cl. A61K 31/275; A61P 25/00; A61K 31/12

(52) U.S. Cl. 514/619; 514/676

(57) ABSTRACT

The invention provides a method for the treatment of certain psychiatric disorders using entacapone, tolcapone and other COMT inhibitor drugs or MB-COMT inhibitor compounds. The method is effective in particular for improving positive and negative symptoms of Schizophrenia (SCZ), major depression, the depressive phase of Bipolar Disorder (BD) and substance dependency. The method can also be used as a treatment to combat cravings associated with abuse of alcohol, opiates, cocaine, marijuana, amphetamines and Tobacco addiction. In addition to these diseases it is useful for the treatment of ADD/ADHD, cognitive enhancement in head injuries and dementias.

### CROSS REFERENCES TO RELATED APPLICATIONS

The present application claims priority of U.S. Provisional Application No. 60/785,097, filed Mar. 23, 2006. The entire contents of the above application are incorporated herein by reference.

### TECHNICAL FIELD

The present invention relates to the use of a composition comprising a COMT inhibitor drug for treatment of schizophrenia, bipolar disorder, addictive behavior and other conditions.

### LITERATURE CITED

Abdolmaleky, H M., S. Thiagalingam and M. Wilcox (2005a). Genetics and epigenetics in major psychiatric disorders: dilemmas, achievements, applications and future scope. Am J. Pharmacogenomics. 5: 11175-2203.
Abdolmaleky H M, Cheng K H, Faraone S V, Wilcox M, Glatt S J, Gao F, Smith C L, Shafa R, Aeali B, Carnevale J, Pan H, Papageorgis P, Ponte J F, Sivaraman V, Tsuang M T, Thiagalingam S. Hypomethylation of MB-COMT promoter is a major risk factor for schizophrenia and bipolar disorder. Hum Mol Genet. 2006 Nov. 1; 15(21):3132-45. Epub 2006 Sep. 19.
Bertolino, A., Caforio, G., Blasi, G., De Candia, M., Latorre, V., Petruzzella, V., Altamura, M, Nappi, G., Papa, S., Callicott, J. H., et al. (2004) Interaction of COMT (Val(108/158)Met) genotype and olanzapine treatment on prefrontal cortical function in patients with schizophrenia. Am. J. Psychiatry 161, 1798-1805.
Blasi, G., Mattay, V. S., Bertolino, A., Elvevag, B., Callicott, J. H., Das, S., Kolachana, B. S., Egan, M. F., Goldberg, T. E., Weinberger, D. R. (2005) Effect of catechol-O-methyltransferase val158met genotype on attentional control. J. Neurosci. 25, 5038-5045.
Bruder, G. E., Keilp, J. G., Xu, H., Shikhman, M., Schori, E., Gorman, J. M., Gilliam, T. C. (2005) Catechol-O-Methyltransferase (COMT) Genotypes and Working Memory: Associations with Differing Cognitive Operations. Biol. Psychiatry. 58, 901-907.
Chen X, Wang X, O'Neill A F, Walsh D, Kendler K S (2004). Variants in the catechol-o-methyltransferase (COMT) gene are associated with schizophrenia in Irish high-density families. Mol Psychiatry. October; 9(10):962-7.
De Frias, C. M., Annerbrink, K., Westberg, L., Eriksson, E., Adolfsson, R., Nilsson, L. G. (2005) Catechol O-methyltransferase Val158Met polymorphism is associated with cognitive performance in nondemented adults. J. Cogn. Neurosci. 17, 1018-1025.
Diaz-Asper, C. M., Weinberger, D. R., Goldberg, T. E. (2006) Catechol-O-methyltransferase polymorphisms and some implications for cognitive therapeutics. NeuroRx. 3, 97-105. Review.
Eley T C, Tahir E, Angleitner A, Harriss K, McClay J, Plomin R, Riemann R, Spinath F, Craig I (2003). Association analysis of MAOA and COMT with neuroticism assessed by peers. Am J Med Genet. July 1; 120B(1):90-6
Galderisi, S., Maj, M., Kirkpatrick, B., Piccardi, P., Mucci, A., Invernizzi, G., Rossi, A., Pini, S., Vita. A., Cassano, P., et al. (2005) Catechol-O-methyltransferase Val158Met polymorphism in schizophrenia: associations with cognitive and motor impairment. Neuropsychobiology 52, 83-89.
Glatt S J, Faraone S V, Tsuang M T. (2003a) Association between a functional catechol O-methyltransferase gene polymorphism and schizophrenia: meta-analysis of case-control and family-based studies. Am J. Psychiatry. March; 160(3):469-76.
Jia, C. X., Zhao, Z. T., Hu, M. H., Gao, L. J., Wang, X. T. (2005) A paired case-control study on related factors to attempted suicide. Zhonghua Liu Xing Bing Xue Za Zhi. 26, 339-343.
Lachman H M, Papolos D F, Saito T, Yu Y M, Szumlanski C L, Weinshilboum R M (1996) Human catechol-O-methyltransferase pharmacogenetics: description of a functional polymorphism and its potential application to neuropsychiatric disorders. Pharmacogenetics. 6(3):243-250.
Massat, I., Souery, D., Del-Favero, J., Nothen, M., Blackwood, D., Muir, W., Kaneva, R., Serretti, A., Lorenzi, C., Rietschel, M., et al. (2005) Association between COMT (Val158Met) functional polymorphism and early onset in patients with major depressive disorder in a European multicenter genetic association study. Mol. Psychiatry. 10, 598-605.

Mattay, V. S., Goldberg, T. E., Fera, F., Hariri, A. R., Tessitore, A., Egan, M. F., Kolachana, B., Callicott, J. H., Weinberger, D. R. (2003) Catechol O-methyltransferase val158-met genotype and individual variation in the brain response to amphetamine. Proc. Natl. Acad. Sci. USA. 100, 6186-6191.
Moreau, J. L., Borgulya, J., Jenck, F., Martin, J. R. (1994) Tolcapone: a potential new antidepressant detected in a novel animal model of depression. Behav. Pharmacol. 5, 344-350.
Rosa A, Peralta V, Cuesta M J, Zarzuela A, Serrano F, Martinez-Larrea A, Fananas L (2004). New evidence of association between COMT gene and prefrontal neurocognitive function in healthy individuals from sibling pairs discordant for psychosis. Am J. Psychiatry. June; 161(6):1110-2.
Sadock B, Sadock V (2000): Kaplan and Sadock's Comprehensive Textbook of Psychiatry, 7 ed, Vol 1. Philadelphia, Pa.: Lippincott Williams & Wilkins, pp 236-272 and 1330-1395.
Shifman S, Bronstein M, Sternfeld M, Pisante A, Weizman A, Reznik I, Spivak B, Grisaru N, Karp L, Schiffer R, Kotler M, Strous R D, Swartz-Vanetik M, Knobler H Y, Shinar E, Yakir B, Zak N B, Darvasi A. (2004) COMT: a common susceptibility gene in bipolar disorder and schizophrenia. Am J Med Genet B Neuropsychiatr Genet. 1; 128(1):61-64.
Stefanis, N. C., van, Os. J., Avramopoulos, D., Smyrnis, N., Evdokimidis, I., Stefanis, C. N. (2005) Effect of COMT Val158Met Polymorphism on the Continuous Performance Test, Identical Pairs Version: Tuning Rather Than Improving Performance. Am. J. Psychiatry. 162, 1752-1754.
Takahashi S, Ohtsuki T, Yu S Y, Tanabe E, Yara K, Kamioka M, Matsushima E, Matsuura M, Ishikawa K, Minowa Y, Noguchi E, Nakayama J, Yamakawa-Kobayashi K, Arinami T, Kojima T (2003). Significant linkage to chromosome 22q for exploratory eye movement dysfunction in schizophrenia. Am J Med Genet. November 15; 123B(1):27-32.
Tasman, Key, Lieberman (2003): Psychiatry. West Sussex, John Wiley & Sons, Ltd., Second Edition, Volume 1, pp 254-272.
Tunbridge, E. M., Bannerman, D. M., Sharp, T., Harrison, P. J. (2004) Catechol-o-methyltransferase inhibition improves set-shifting performance and elevates stimulated dopamine release in the rat prefrontal cortex. J. Neurosci. 24, 5331-5335.
Williams N M, Norton N, Williams H, Ekholm B, Hamshere M L, Lindblom Y, Chowdari K V, Cardno A G, Zammit S, Jones L A, Murphy K C, Sanders R D, McCarthy G, Gray M Y, Jones G, Holmans P, Nimgaonkar V, Adolfson R, Osby U, Terenius L, Sedvall G, O'Donovan M C, Owen M J (2003). A systematic genomewide linkage study in 353 sib pairs with schizophrenia. Am J Hum Genet. 73(6):1355-67.

### BACKGROUND

More than five million people in the USA and 120 million worldwide suffer from Schizophrenia (SCZ) and bipolar disorder (BD). SCZ is marked by positive symptoms (e.g. hallucinations, delusions and disorganized behavior) and negative symptoms (e.g. lack of interest, attention and volitions). The main problem of patients with BD is circular mood changes raging from severe depression to severe mania with or without psychotic features. These illnesses tend to be chronic/progressive and are among the most severe forms of psychiatric disorders that elicit overlapping cognitive deficits (Tasman et al., 2003; Sadock and Sadock 2005). Positive symptoms often are controlled with antipsychotic drugs treatment; however, depression in BD and negative symptoms in SCZ remains a major dilemma in psychiatry. Antidepressant treatment may lead to manic flair up (shifting to the manic phase), while lack of treatment and stagnation in the depressive phase may result in suicide or functional deficits. The same is true for negative symptom of SCZ, i.e. negative symptoms tend to persist and can produce functional failure and a dependent life style. Although clozapine and other atypical anti-psychotic drugs are partially useful for the treatment of negative symptoms, their common side effects such as agranulocytosis, sedation, weight gain, hyper-lipidemia and hyperglycemia limit their applications (Tasman et al., 2003; Sadock and Sadock 2005). Thus, alternative medications are needed for relief of negative as well as depressive symptoms of SCZ and BD.

The treatment of these diseases represents one of the highest medical costs in western society with 2% of the gross national product spent on SCZ alone (Sadock and Sadock 2005). Long-term, daily maintenance has created a very large market: sales in 2003 were more than $6 billion for atypical antipsychotic medications. Market share leaders for treatment of SCZ include Zyprexa from Eli Lily (38% prescription share 42% of revenue, and $2.5 billion income) and Risperdal from Johnson & Johnson (37% prescription share, 23% of revenue, $1.4 billion income). Medicare has put pressure on the high cost of Zyprexa ($9-$10 per day) and in some cases limited reimbursement to specific diagnosis.

Patients with SCZ and BD exhibit hypofrontality, attentive problems and executive dysfunctions with associated lack of responses and failures to suppress inappropriate ones. There is strong evidence that frontal lobe dopamine (DA) deficiency is responsible for these symptoms in SCZ and BD. DA is involved in several brain activities including; attention, executive memory, hedonic activities, natural rewards, and biological activities such as cell signaling. Most of these effects are mediated through the DA receptors (i.e. DRD1 and DRD2) which act on other cell signaling pathways (Tasman et al., 2003; Sadock and Sadock 2005; Abdolmaleky et al., 2005; Abdolmaleky et al., 2006). Additionally, COMT and MAOA, the DA catabolizing enzymes, and dopamine transporter (DAT1), involved with reuptake of DA from the synaptic cleft, have significant roles in dopaminergic signal transmission (FIG. 1). Our previous work indicated that most components of brain dopaminergic system are related to the pathogenesis of SCZ, BD, ADHD and substance dependency (Abdolmaleky et al., 2005; Abdolmaleky et al., 2006).

Clearly, there is a need for additional medications efficacious for the treatment of these disorders, and especially for medications that suppress or eliminate the recurrent unwanted, intrusive, or involuntary thoughts, perceptions and behaviors characteristic of the disorders. Such medications might also be used to reduce such symptoms when they occur as part of another psychiatric syndrome or when they are incidental to a neurological disorder.

### SUMMARY OF THE INVENTION

We disclose here a method for the treatment of certain psychiatric disorders using entacapone, tolcapone and other COMT inhibitor drugs.

We disclose a method of treating symptoms associated with a psychiatric disorder, comprising administering to a patient a pharmacologically effective dose of a composition comprising a COMT inhibitor or MB-COMT inhibitor drug or a pharmaceutically acceptable salt thereof.

The method is effective in particular for improving negative symptoms of SCZ, augmentation of effect of anti-psychotics in treatment of positive symptoms of SCZ, in treatment of major depression, the depressive phase of BD and substance dependency. It can be used as a treatment to combat cravings associated with abuse of alcohol, opiates, cocaine, marijuana, amphetamines.

The method is also effective for the treatment of tobacco addiction and the weight gain/food cravings associated with quitting smoking or the use of antipsychotics.

In addition to other DA deficiency-related diseases such as ADD/ADHD, it can be used for cognitive enhancement in head injuries and dementias.

COMT inhibitor drugs have a beneficial effect in ill individuals if the principle cause of negative symptom formation is due to frontal lobe hypo-DAergic activity from MB-COMT over activity. Thus, COMT inhibitors are expected to be more useful in individuals with hypo-methylated MB-COMT promoter and/or Val/Val and Val/Met genotype than those with Met/Met genotype. Patients with COMT valine genotype are more resistant to typical and atypical anti-psychotic drugs (Bertolino et al., 2004).

Entacapone and tolcapone are oral used peripheral and central selective COMT inhibitor drugs that increase the brain DA transmission in the presence of relevant stimulations. These drugs are used for Parkinson's disease for more than a decade. Tolcapone is a safe and a well tolerated drug with no major side effects, although three cases of hepatic failure was reported in Europe in the early years of use. Since the drug treatment is accompanied with liver enzyme monitoring, no new hepatic failure has been reported. Entacapone penetrates the blood brain barrier at a level half that of about tolcapone but, has much better safety profile.

We discovered a “Triad of Deficit” in postmortem human brain studies of BD and SCZ that could be a potential explanation of hypofrontality. This Triad consists of RELN under-expression, COMT over-expression and defective DRD2 gene expression in the frontal lobe concurrent with MAOA hyperactivity. Our data indicated that RELN and DRD2 dysfunction is secondary to MB-COMT hyperactivity. Based on this discovery we incepted treatment with COMT inhibitor drugs, including but not exclusive to Entacapone or Tolcapone.

### BRIEF DESCRIPTION OF THE DRAWINGS

The foregoing and other objects, features and advantages of the invention will be apparent from the following more particular description of preferred embodiments of the invention.

FIG. 1 is an illustrative view of neuronal factors and receptors affected in SCZ and BD; and

FIG. 2 is a graphical illustration showing the expression profile of DRD1, DRD2 and RELN versus MB-COMT in high and low MB-COMT expressive groups, respectively.

### DETAILED DESCRIPTION OF THE INVENTION

COMT is also one of the most intensively investigated genes in psychiatric illness because of its ability to regulate the homeostatic levels of the neurotransmitter DA in synapses. It is also interesting that the COMT gene is localized to chromosome 22q11.21, a region identified by several genetic studies as linked to SCZ, BD, ADHD and substance dependency (Williams et al, 2003; Takahashi et al., 2003). COMT has two known isoforms: membrane-bound COMT (MB-COMT) and soluble COMT (S-COMT) transcribed from two different promoters. MB-COMT is the predominant form involved in the degradation of synaptic DA in the human brain (Lachman et al., 1996). COMT over activity is related to an increased rate of DA degradation that can lead to the social withdrawal personality trait, disturbance in attention, executive cognition, and working memory performance (Eley et al., 2003; Rosa et al., 2004). MB-COMT has a functional polymorphism at codon 158 and the valine-coding allele of this polymorphism codes for an enzyme with approximately three times higher physiological activity than the methionine containing allele (Lachman et al., 1996). Several studies showed that increased DA degradations arising from COMT hyperactivity is associated with disturbances in attention, executive cognition, and working memory performance in normal populations and schizophrenic patients and that these effects may even be progressive over time (Eley et al., 2003; Rosa et al., 2004; Blasi et al., 2005; Bruder et al., 2005; De Frias, 2005; Galderisi et al, 2005; Stefanis et al., 2005). In contrast, the hypoactive allele (Met) of the gene is associated with less variability in reaction time and greater stability in performance (Stefanis et al, 2005). Additionally, based on ours (Abdolmaleky et al., 2006) and others studies, the Val allele of COMT Val158Met polymorphism is linked to SCZ, early onset SCZ, BD, early onset major depressive disorder and suicide confirming that in addition to SCZ and BD, depressive symptoms and suicide are associated with the hyperactivity of COMT gene (Glatt et al, 2003; Chen et al., 2004; Shifman et al., 2004; Massat et al., 2005; Jia et al., 2005; Tunbridge et al., 2006, Diaz-Asper et al., 2006).

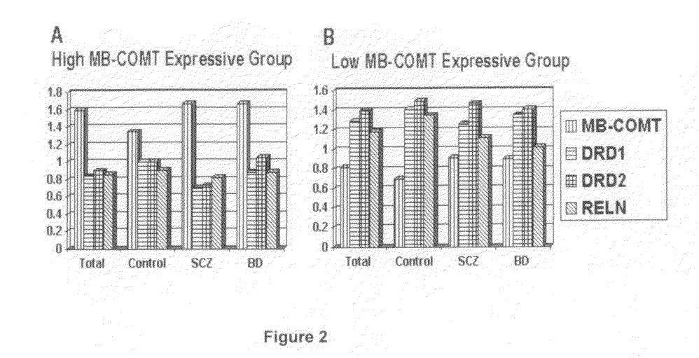

We have discovered a highly significant level of hypomethylation of the MB-COMT promoter in post-mortem brains of patients with SCZ and BD compared to control subjects (4). In addition, the degree of DNA methylation was inversely correlated with transcript quantities as determined by quantitative real-time PCR using primers that exclusively could amplify the MB-COMT isoform transcript, the predominant form involved in the degradation of synaptic DA in the human brain (Abdolmaleky et al., 2006). These results reveal that hyper-activity of the COMT gene confers liability for SCZ and mood disorders through rapid degradation of DA in the synaptic cleft. This will produce a hypo-DAergic state in the frontal lobe which might account for the reported frontal hypo-activity and known SCZ-associated problems in attention, desire, hedonic and social activity, cognitive processes and working memory. It appears that MB-COMT hyper-expression in the frontal lobe of human brain is associated with decrease in the expression of DRD1, DRD2 and RELN (FIG. 2). We also discovered a significant association between the presence of valine (hyperactive allele) of COMT and RELN promoter hypermethylation, a hypo-expressed gene in SCZ and BD.

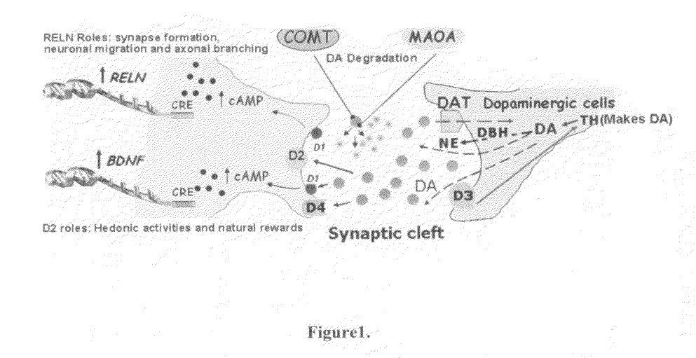

Referring to FIG. 1, which shows how neuronal factors and receptors are affected in SCZ and BD, DAergic neurons release DA that is involved in several brain functions, including attention, executive memory, desire, hedonic activities and natural rewards mediated by a network of cell signaling events. Most of these effects are likely to be downstream of D1 and D2 like receptors. As was shown in details for BDNF, some of these effects are mediated by D1 receptors acting on the intracellular c-AMP machinery influencing the methylation status of core cytosine of CRE in the promoter of the effector genes, while neuronal inactivation leads to the CRE cytosine methylation.

Our studies showed that COMT over-activity (due to its promoter hypo-methylation associated over-expression or the hyperactive polymorphic valine allele at the Val158Met polymorphism) leading to rapid DA degradation and DA deficiency in the synaptic cleft could have similar effects on CRE methylation status of RELN promoter as well as the methylation/expression levels of DRD1 and DRD2 genes. Our recent studies showed that MAOA is also over-expressed in SCZ and BD which intensify the effects of MB-COMT over-expression.

We found a very low level of DAT1 expression in the frontal lobe both in controls and patients implying that the effects of MB-COMT and MAOA could not be affected or compensated by DAT1 activity in this brain region. The pre-synaptic D3 receptors (DRD3) modulate TH expression and synaptic DA level through an unknown mechanism. Likely, under-stimulation of pre-synaptic DRD3 influenced by synaptic DA deficiency may affect the activation of CRE in the TH promoter and its subsequent cytosine methylation and expression status. DA is also an intermediate product for biosynthesis of nor-epinephrine (NE), an important neurotransmitter/modulator involved in mood regulation and neurodevelopment, by mediation of DBH. Hence, this could leads to a change in production of DA and NE and subsequent dysfunction of DA/NE effector genes. We found that DRD1, DRD2, DRD3 and DRD4 expressions are inversely correlated with MB-COMT expression, thus MB-COMT over-expression could impair the DA/NE signaling events and end effects. These events may also influence developmental/primary or state-dependent/secondary promoter methylation of BDNF, RELN and other genes leading to hypo-expression of these genes affecting neuronal outgrowth, positioning synapse formation and other functions. An underdevelopment of neuronal connections and synapse structures may result in further insufficiencies in the scarcity of sub-synaptic elements such as DA and other receptors.

In addition, RELN and the DRD2 promoter regions were also significantly hyper-methylated in SCZ and bipolar patients who had hypo-methylated COMT promoter vs. control subjects (p=0.001), discovering that a DA deficiency resulted from the COMT hypomethylation, or over active allele, may influence promoter methylation and/or expression of DRD2, RELN and possibly other genes. Most recently, we also discovered that the expression of MB-COMT is inversely correlated with DRD3 and DRD4 expression. Additionally, MAOA (the second enzyme responsible for DA degradation in the synapses) was over-expressed in SCZ and BD which intensified the effects of MB-COMT over-expression. We found a very low level of DAT1 expression in the frontal lobe both in controls and patients implying that the effects of MB-COMT and MAOA could not be affected or compensated by DAT1 activity in this brain region. Collectively, these observations strongly confirmed that a DA deficiency resulted from the COMT hypo-methylation, or over active allele, may influence promoter methylation and/or expression of DRD1-4, RELN and possibly other genes and these effects are aggregated due to a concurrent over-expression of MAOA. Considering the role of RELN in neuronal migration and synapse formation, individuals with a dysfunctional RELN signaling will have an under-developed synapse structure. As a result, sub-elements of these synapses, such as DA and other receptors, are less than normal individuals. In this situation an adaptive fine-tuning of the MB-COMT promoter methylation/expression could have a critical role to ensure an appropriate level of DAergic tone in order to compensate the low levels of DA receptors in the synapses. Although, normal individuals showed a strong tendency for such adaptive fine-tuning, a large portion of the patients with SCZ and BD failed to employ this remedy. As a result, not only the structures of synapses are under-developed and endure from the insufficient abundance of DA receptors, an adequate DAergic tone is not also provided due to the hyper-activity of MB-COMT.

The findings above reveal that MB-COMT is a unique payer in the frontal lobe of human brain. Over-activity through a cascade of events has a significant role in etio-pathology of negative symptoms of SCZ as well as depressive symptoms of unipolar and BD. Hence, COMT inhibitor drugs are useful in patients with the Val/Val (and Val/Met) genotype and/or a hypomethylated COMT promoter. Animal studies showed that Tolcapone a mix peripheral and central COMT inhibitor drug significantly improves extra dimensional set shifting performance mediated by prefrontal cortex catecholamines, including DA (Tunbridge et al., 2004). COMT inhibition elevates extra-cellular DA under conditions of evoked catecholamine release. In the caudate COMT inhibitor treatment reduces DA metabolites as it prevents DA catabolism in the synapses. As, the COMT 158 Met allele (under-active) is associated with better performance and greater efficiency in prefrontal cortex function tests in SCZ and improvement of cognitive symptoms by atypical antipsychotic drugs such as clozapine and olanzapine are related to prefrontal cortex DA release (Tasman et al., 2003; Sadock and Sadock 2005), tolcapone which augments the DA release in the frontal cortex has therapeutic effects, alone, or in combination with antipsychotic drugs.

In animal studies, COMT inhibitor drugs prevented the stress-induced anhedonic state, improved the prefrontal cortex performance and potentate clozapine induced extracellular DA release (Moreau et al., 1994). Tolcapone a mix peripheral and central COMT inhibitor drug significantly improved extradimensional set shifting performance mediated by prefrontal cortex catecholamines in rats and primates (Tunbridge et al., 2004). In human studies, other DAergic drugs such as amphetamine enhanced the efficiency of prefrontal cortex functions in working memory tasks in subjects with the Val/Val genotype (Mattay et al., 2003), however these drugs are not safe for long term use (Tasman et al., 2003; Sadock and Sadock 2005). Theoretically, MAOA inhibitors could be used as an alternative drug. However, our analyses showed that the level of expression of MAOA in the human frontal lobe is less than half compared to the MB-COMT expression. Thus, unlikely MAOA inhibitors could provide sufficient DAergic tone for the frontal lobe. Practically, MAOA inhibitors are known to be not effective for these situations (Tasman et al., 2003; Sadock and Sadock 2005). Furthermore, prescription of these drugs requires a restricted diet (e.g. complete avoidance of alcohol, vinegar, chocolate, coffee, cheese etc.) (Tasman et al., 2003; Sadock and Sadock 2005) that is not ensured in psychotic or patients with drug dependency, thus are not safe. Hence, COMT inhibitor drugs would be more appropriate to provide sufficient DAergic tone for the frontal lobe in these patients.

Referring to FIG. 2, consistent with the promoter methylation status, expressions of RELN, DRD1 and DRD2 appear to be correlated, but are inversely correlated with the MB-COMT expression both in controls and the patients a well as in the total sample. As a result MB-COMT hyper-expression could be associated with hypo-activity of DAergic neurotransmission and RELN hypo-expression in the frontal lobe. Panel A and B show the expression profile of DRD1, DRD2 and RELN versus MB-COMT in high and low MB-COMT expressive groups, respectively.

Entacapone is a catechol-O-methyl transferase inhibitor for the treatment of Parkinson's disease. When administered in conjunction with dopaminergic agents such as L-DOPA, entacapone increases the bioavailability of these compounds by facilitating their passage across the blood-brain barrier. It is a member of the class of nitrocatechols. The most frequent undesirable effects caused by entacapone relate to the increased effects of L-DOPA, such as involuntary movements (dyskinesias). These occur most frequently at the beginning of entacapone treatment. Others common side effects are gastrointestinal problems, including diarrhea, nausea and abdominal pains. The substance may cause urine to turn reddish-brown. This is a harmless side effect and is not a cause for concern. In studies with entacapone, some people have reported experiencing a dry mouth. Entacapone is developed by Orion Pharma and marketed by Novartis under the trade names Comtan® and Stalevo® in the United States. Stalevo® is a medication that contains carbidopa, levodopa (active ingredients in Sinemet®), and entacapone (active ingredient in Comtan®).

Entacapone and tolcapone are oral used COMT inhibitor drugs that increase the brain DA transmission in the presence of relevant stimulations. Entacapone penetrates the blood brain barrier at a level half that of about tolcapone but, has much better safety profile. The use of entacapone should avoid any adverse event. In addition, before drug testing, patients should undergo a physical examination that will include an EKG, CBC, liver functions, Urine Analysis, urine Pregnancy Test for female subjects, and a Urine Toxic Screen. Those patients who have any liver diseases or medical problem which may interfere with the drug efficacy or would put them at risk should be excluded.

Although, currently a COMT inhibitor drug specific for MB-COMT isoform (the main form in the human brain) in not available in the market, in the future these drugs (MB-COMT specific inhibitors) could be developed in the form of patch, spray or long acting injections.

This new therapeutic application was determined in patients (Men/women) in single-drug and combination drug therapy. 100 patients were treated at a dose range of 3-10-mg/kg day (50 mg/day-800 mg/day) orally for period of 12 weeks and continued for as long as patients consented averaged 12 months or more.

The medicinal products which are useful in the treatment of these diseases consist of a COMT inhibitor drugs or MB-COMT inhibitors or a pharmaceutically salt thereof either alone or in the form of a composition in which it is combined with any other pharmaceutically compatible product, which may be inert or physiologically active. These medicinal products may be used orally, topically, parenterally or rectally.

Pharmaceutical compositions of the compound of the present invention or its salts are produced by formulating the active compound in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses. Some examples of suitable pharmaceutical carriers, including pharmaceutical diluents, are gelatin capsules; sugars such as lactose and sucrose; starches such as corn starch and potato starch, cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations. The compositions of the invention can also contain other components such as coloring agents, flavoring agents, and/or preservatives. These materials, if present, are usually used in relatively small amounts. The compositions can, if desired, also contain other therapeutic agents.

The percentage of the active ingredients in the foregoing compositions can be varied within wide limits, but for practical purposes it is preferably present in a concentration of at least 10% in a solid composition and at least 2% in a primary liquid composition. The most satisfactory compositions are those in which a much higher proportion of the active ingredient is present.

Routes of administration of the subject compound or its salts are oral, topical or parenteral. For example, a useful oral dosage is between 100-800 mg per day. A unit dosage form of the instant invention may also comprise other compounds useful in the therapy of neurodegenerative diseases.

As solid compositions for oral administration, tablets, pills, powders (gelatin capsules, wafer tablets) or granules may be used. In these compositions, the active principle is mixed with one or more inert diluents such as starch, cellulose, sucrose, lactose or silica. These compositions can also comprise substances other than diluents, e.g. one or more lubricants such as magnesium stearate or talc, a coloring, a coating (dragees) or a lacquer.

As liquid compositions for oral administration, there may be used solutions, suspensions, emulsions, syrups and elixirs which are pharmaceutically acceptable, containing inert diluents such as water, ethanol, glycerol, vegetable oils or liquid paraffin. These compositions can comprise substances other than diluents, e.g. wetting, sweetening, thickening, flavoring or stabilizing products.

The compositions for parenteral administration may be sterile suspensions, emulsions or nonaqueous solutions. As a solvent or vehicle, water, propylene glycol, a polyethylene glycol, vegetable oils, especially olive oil, injectable organic esters, e.g. ethyl oleate, or other suitable organic solvents may be employed. These compositions can also contain adjuvants, especially wetting agents, tonicity regulators, emulsifiers, dispersants and stabilizers. The sterilization can be carried out in several ways, e.g. by aseptic filtration, by incorporating sterilizing agents in the composition, by irradiation or by heating. The compositions can also be prepared in the form of sterile solid compositions which can be dissolved in an injectable sterile medium immediately prior to administration.

The compositions for rectal administration may be suppositories or rectal capsules containing, apart from the active product, excipients such as cocoa butter, semi-synthetic glycerides or polyethylene glycols.

The drug may also be in any topical preparation including rubbing gel or patch form. Variations would be known to those skilled in the art. Generally speaking, the doctor will determine the appropriate dosage in accordance with the age, weight and all other factors specific to the subject to be treated.

### Clinical Studies:

Our clinical studies on more than 100 patients with SCZ, BD, Major depression and drug dependency showed that entacapone (Comtan, 50-800 mg per day), an inhibitor of COMT activity could improve negative and depressive symptoms in more than 60% of cases. The drug efficacy was evaluated using the CGI (Clinical Global Impression) scale. This is a simple tool that is clinically understandable, has proven to be a robust measure of efficacy in drug treatment trial and is highly sensitive to change of clinical status. During the study we found no indication of serious events such as physical, neurological or psychiatric adverse effects. Not only psychotic patients, but also a vast majority of patients with drug dependency, obesity and depression were benefited from this treatment in our clinical trials.

### ILLUSTRATIVE EXAMPLES

### Two Cases of SCZ

Case #1

Treatment of positive symptoms of SCZ: 58 years old unemployed divorced white male, a former engineer with history of diabetes (on Insulin treatment), with intractable symptoms of suicidal command auditory hallucination “over dose on Insulin”, along with visual hallucinations seeing “a black tube” who has failed response to host of conventional and atypical antipsychotics (monotherapy or in combination) as well as no response to treatment with clozapine (as monotherapy and combination with all atypical antipsychotic drugs and number of typical drugs with and without addition of lithium, Divalproex Sodium and Lamotrigine all in combination or as monoadjunctive therapy. He has had number of hospitalizations following serious suicide attempts as result of the hallucinations. He has been seen for medication management and therapy on weekly basis for the past 8 years. Finally due to lack of response his medications had been trimmed down to clozapine monotherapy for numbers of weeks when entacapone 100 mg/day was added which was increased to 200 m/day after 2 days and by day 4 into treatment he became absolutely free of hallucinations and has maintained the status for the past 7 months.

Case #2

Treatment of Negative symptoms of SCZ: 50 years old unemployed college drop out single white male with diagnosis of SCZ who his positive symptoms of paranoid delusion and hallucinations, has been stabilized with combination of Clozapine, Ziprasidone and Lamotrigine. He had not need any hospitalization for 18 months and has been leading eventful life. He yet has been suffering the negative symptoms of SCZ complaining of poor short-term memory, poor attention span, inertia, lack of motivation, social isolation, and withdrawn behavior. “I want to come out of my shell but it is very hard”. He was put on Entacapone 100 mg/day by week 2, he reported that he felt more emotional energy, was able to make plan and not to procrastinate, felt motivated to do things and no longer felt overwhelmed by “flood of daily chores and things he was supposed to do, felt he was able to focus on one thing at a time especially when he was asked to do complex task, felt more confidence and therefore became more forthcoming in conversations, no longer felt scared of pitching in a social conversation. He started to brush his teeth, shave, shower and do his daily hygiene without being promoted by his parents. Through the course of treatment his dose was adjusted to 400 mg/day. He was able to leave his house go to the town library, found a self help book on SCZ, utilized that as a focus point in therapy learned to his own laundry, is getting cooking lessons from his mother and is participating in balancing his check book and his finances with help of his father. He continues to maintain the positive effect of entacapone treatment and he has not shown any hypo manic episode in the past 8 months. He continues to be active in his bi-weekly treatment.

### A Case of BD:

39 years old married white male, a hedge fund investment manager with history of BD who has had temporary partial response to antidepressants but mainly resulting in exacerbation symptoms of anxiety, relapse to a depression or cycling to mania. He was intolerant of atypical antipsychotics with potential antidepressant effect (Ziprasidone, Aripiprazole and Quetiapine) either as mono therapy or combined with Lithium or Lamotrigine. Lithium was kept as the main mood stabilizer and because of patient's passive suicidal thoughts (“If I had a gun I would shoot myself”) and Lorazepam to help with the anxiety. He reported oil Lithium his “up and down swings were better” but complained his depression was not resolved and he was not happy. He also complained of lack of motivation, trouble with concentration, getting easily overwhelmed with his job, inability to feel joy, overall lack of interest in everything, not knowing what to do with himself, gave up on all hobbies, feeling being all consumed, excessive day time napping, feeling bored especially when his time would be unstructured on week end, loss of his spontaneity and resorting to procrastination, He went to an exotic vacation in Caribbean with his wife with the hope to bounce back. But as he reported he was not able to enjoy his vacation, complained feeling “being stuck at the low bottom and strongly believed he would never come out of it”. He had started to do Marijuana with the hope to improve his mood. He was preoccupied with getting a gun and shoots himself. He was started on Entacapone at 100 mg/day for 7 days and was increased to 200 mg/day. On next visit at week 4, he reported, he had a “good month without being manic, had fun and no panics”, he has been able to take care of things at work, restarted his hobbies, has been reading books, felt confident to make critical decisions despite the fact that they had a difficult financial market, played his guitar at his leisure time, had positive out-look, had not thought about “gun” any more, as the financial situation has been deteriorated had been able to be proactive and sent his resume for other job opportunities, and reported overall he was no longer depressed or anxious, did not require lorazepam, had no longer any negative thoughts, and felt “healthy”. He stated that he was back to the way he used to feel as a “healthy adult before he ever got sick with the BD”, and was able to recognize depression or anxiety in others. His Entacapone was increased to 400 mg/day to assure sustaining of his improvement. He has maintained the effect so far after 12 months of follow up.

### Six Cases of Drug Dependency:

Case #1

A Dependant to Marijuana: 39 years old married black male with history of 5 years incarceration for violent behavior under influence of substances who reports being sober from alcohol, his last cocaine use was 17 years ago, his last heroin/opiate use was 3 years ago but complained he has been using daily Marijuana since age 12 and has not been able to manage one day without it, and he smokes up to 10 times a day. He had been court mandated to stop Marijuana despite his best effort he had not been able to comply and feared he would finally be caught in drug testing and would be incarcerated again. He was diagnosed with co-morbid of BD and he was treated with Lithium, Divalproex Sodium, aripiprazole and Bromocriptine with good response in mood stabilization and management of paranoia and violent tendencies yet he complained of sever marijuana craving despite taking bromocriptine 10 times a day. Out of frustration he stopped his medications but agreed to retake aripiprazole as monotherapy for mood and bromocriptine so frequent during the day in lieu of Marijuana use. He had one more positive urine test but his probation officer gave him a break because his good conduct. He complained of sever craving for Marijuana. Entacapone was started at 100 mg/day and by day 2 of treatment his craving for marijuana ceased completely at the end of the week 3 his entacapone was increased to 200 mg/day and he remained free of any cravings 7 months into follow ups. He remained totally abstinent and drug free. He moved to another town and is lost to follow up.

Case #2

A Cocaine and Alcohol dependant: 42 years old single unemployed white male with working diagnosis of SCZ who has been stabilized on combination of clozapine and aripiprazole has not been able to abstain from alcohol and cocaine despite strong structure of day program daily AA, weekly counseling at drug treatment center, and close family monitoring. His drug of choice was cocaine and he used alcohol to “bring him down if too high on cocaine”. He complained he could not sit still in AA meeting and had hard time to concentrate due to strong cocaine craving. He was started on entacapone 100 mg/day for 2 days and increased to 200 mg/day by day 4. He reported that he had no craving for cocaine or alcohol, he was able to focus and did not feel depressed and had no longer feelings of guilt. By the end of week 3 he had no longer “drug dreams”. He continued with his supportive drug treatment. He remained abstinent for the next 10 months. But when at the month 10 of follow up, his main care provider (his mother) died he stopped entacapone and he relapsed on cocaine, he stopped all

Case #3

An Alcohol Dependent (combination therapy): 48 years old married white female a shop assistant with strong family history of alcoholism who began drinking age 15 and soon after developed a strong drinking habit (quart of vodka and 2 bottle of cheap wine). Through course of years she had sought various forms of treatment including more than 18 detox hospitalization, number of rehabs, half way houses and sober houses, but the longest period of time had remained sober was 3 months when she was incarcerated for violation of parole on the charges of driving under influence of alcohol. Her strive for sobriety has demonstrated failure repeatedly, she had had delirium Tremens and withdrawal seizures. Her psychiatrist dismissed her when she relapsed within few days of her 13th discharge. She started treatment in our clinic in 2003, when she was put on Topiromate. She was diagnosed with the co-morbidity of mood disorder NOS and received pharmacologic treatment, (failed Divalproex sodium, buspirone, olanzapine, s-citalopram and carbamazepine). Finally her mood became stabilized on combination of (lamotrigine and aripiprazole), but she was not able to attain any meaningful episode of sobriety. In the next 3 years as 1Topiromate was continued to assure seizure protection and to address for concern over weight management. She failed an adequate trial of Accomprosate. She was intolerant of Naltrexone and refused Disulfiram. During these treatment trials she relapsed and failed treatment repeatedly despite her avid AA participation. Entacapone 200 mg/day was started at the completion of a 7 day in-patient detox. She reported by day 7 that felt a relief, her craving stopped and she felt confident that she could stay in the half way house and continue with the hospital after care treatment plan. The Entacapone was increased to 400 mg/day to secure the response. Five months into her sobriety she complained of being tired, “stressed out” and depressed, her Entacapone was increased to 600 mg/day, she felt the depression was lifted and her energy became satisfactory. She has remained active in AA, and has maintained sober for the past 14 months. She reports, she does not have any craving for alcohol at all.

Case #4

An Alcohol Dependence (Entacapone Monotherapy): 49 years old single white female with history of alcohol consumption since age 18, with history of black outs, hand and body tremors as her alcohol would depreciate. She had had one period of sobriety during her pregnancy of 20 years ago. Other than that, she had maintained between 4-7 beers a day habit. She joined the treatment began individual counseling, went through two weeks of detoxification and as she was experiencing symptoms of depression and strong alcohol craving. She was put on Entacapone 100 mg/day for 2 days and increments of 100 mg/day ever 2 days to 400 mg/day. At her next visit, 3 weeks later, she reported the her depression was all resolved, had not had any relapse, was free of any craving, began to eat healthy and exercise regularly. She said she was “100% improved” and felt great! She has been regular with her follow ups in the past 6 months and continues to maintain her sobriety. She remains free of any symptoms of dysphoria and alcohol cravings.

Case #5

A Tobacco dependence: 27 years old single white male with significant history of alcohol and polysubstance abuse (opiates as his drug of choice), who smokes 1½ pack a day smoking habit since age 15. He is currently in Buprenorphine maintenance program. He has been diagnosed with co-morbid of BD for which his mood symptoms are stabilized on combination of Lamotrigine and Aripiprazole. Entacapone was initiated for help prevent multiple relapses lie had on alcohol and opiates initially. As he has managed to comply with the treatment program and has had no relapse on either of alcohol and opiate 60 days. He had expressed his desire to quit smoking and has been taking Entacapone 400 mg three times a day and as result he initially decreased his smoking down to one pack per day and after one week of treatment he quit completely and has been smoke free for the past 60 days. He reports no craving for cigarettes except when some body talk about it and he always reserves one of his Entacapones for that moments and takes 200 mg as per needed basis to over come his instant desire.

Case #6

An Opiate dependence: 45 years old married white male a carpenter, with history of Heroin dependence since age 17, now (snorting 7 bags a day), claims the he stopped IVDA 10 years ago. He once remained clean for 6 years with help of AA/NA, but since his relapse of 5 years ago he has not been able to maintain any meaningful periods of abstinence. He reports he used to be an alcoholic and at age 14 almost died of alcohol poisoning and substituted it with the opiates to control his feelings of anxiety and dysphoria. He joined Buprenorphine detoxification program with the hope to be cleaned in a short while and to be put on Nalterxone implant maintenance program. He was an avid AA/NA participant and worked closely with his sponsor. His family was very supportive, but also demanding of him achieving total abstinence as soon as possible. In the 1 month treatment follow up session of treatment, he reported no craving on opiates as he was on a higher dose of Buprenorphine (16 mg/day). But he resisted any taper of buprenorphine, requesting higher dose of the medicine; complaining of general lack of interest, nervousness, anxious feeling, lethargy, lack of motivation, depression especially in the afternoon and symptom of insomnia as he had been taking all his Buprenorphine dose in the morning and subsequently he had split his morning dose to 8 mg in the afternoon to “feel better”. Lamotrigine was started as it was found that he had been doing Heroin to self treat the aforementioned symptoms and had found heroin as an “energy booster, antidepressant”. He was diagnosed with mood disorder NOS as a co-morbid. On Lamotrigine his mood symptoms improved and his anxiety and insomnia responded to Gabapentin. But he kept complain of lack of energy, which made him crave opiate relapse. He managed not to relapse, in the next 10 months, thanks to his comprehensive support system, but he was not able to taper off Buprenorphine as desired. He also expressed his reluctance about proceeding to drug free state and being kept on Naltrexone implant maintenance, since in the past when he used to be clean he tried Naltrexone tablets, which helped his cravings but did not help his lack of energy, so he was concerned that on Naltrexone he would feel the same and therefore he would be entice to drop out of treatment and relapse on Heroin, since no treatment including Buprenorphine had replaced the Heroin effect for him. Finally, Entacapone 200 mg/day was added to the treatment medication. By week 4 of treatment he was able proceed with decrease in dose of Buprenophine. On week 8 of follow up visit, he reported “his energy was high”, he had no symptoms of depression or anxiety, no craving for the opiates and expressed his desire to taper off Buprenorphine and go on the implant. But on the 12 week visit he requested not to go off the 2 mg/day remaining dose of the Buprenorphine due to number of social and financial stressors recently introduced in his life. His Entacapone was increased to 400 mg/day which helped him not to increase his Buprenorphine dose. His Enacapone was further adjusted in the course of next 5 months up to 400 mg/day. He remained free of opiate craving and symptoms of depression but still lacked enough confidence to take the last step of going off the Buprenorphine. His Entacapone was increased to 400 mg twice a day and after 2 months he managed to go off Buprenorphine, remained opiate free for 10 days and finally received the Naltrexone implant and remained totally abstinent for next 4 months when he transferred his follow up care to his primary care physician.

We demonstrated efficacy of entacapone in acute phase of treatment (12 weeks), with extensions to 12 months or more (depending on the patients desire to continue the treatment follow up). Our data reflects as long as patients continued with the treatment maintained the benefit and as the treatment stopped the benefit was lost. Discontinuation of treatment did not cause any worsening of the clinical condition in comparison to the pre-treatment status. There was no patient who lost the treatment response despite continuation of the treatment. The long-term data demonstrates efficacy of treatment as maintenance

While the foregoing specification teaches the principles of the present invention, with examples provided for the purpose of illustration, it will be understood that the practice of the invention encompasses all of the usual variation, adaptations and/or modifications as come within the scope of the following claims and their equivalents.

What is claimed is:

1. A method of treating symptoms associated with a psychiatric disorder, comprising administering to a patient a pharmacologically effective dose of a composition comprising a COMT inhibitor or MB-COMT inhibitor drug or a pharmaceutically acceptable salt thereof.

2. The method according to claim 1 wherein the disorder is selected from the group consisting of bipolar depression, unipolar depression, ADD/ADHD, schizophrenia, chronic fatigue syndrome, it can be used for cognitive enhancement in head injuries and dementias.

3. The method according to claim 1 wherein the method is used to combat cravings associated with abuse of alcohol, opiates, cocaine, marijuana, tobacco, or amphetamines.

4. The method according to claim 1 wherein the method is used to treat the weight gain associated with quitting smoking, food cravings or the use of antipsychotics.

5. The method of claim 1, wherein said COMT inhibitor drug is selected from the group consisting of entacapone and tolcapone.

6. The method of claim 1 wherein the composition is available in blood.

7. The method of claim 1 wherein the composition is available in the brain.

8. The method of claim 1 wherein the pharmaceutically effective dosage is about 3-10 mg of the drug/kg of body weight equivalent of 50-800 mg/day.

9. The method according to claim 1 wherein the pharmaceutical composition is in unit dosage form, comprising from about 0.05 mg to about 2000 mg of the composition or a pharmaceutically acceptable salt thereof or a pharmaceutically acceptable solvate thereof.

10. The method according to claim 1 wherein the composition or pharmaceutically acceptable salt thereof is administered orally, parenterally or rectally.
